(19) United States
(12) Patent Application Publication
(10) Pub. No.: US 2023/0417672 A1
(43) Pub. Date: Dec. 28, 2023

(54) SINGLE COPY LEVEL DETECTION OF CORONAVIRUSES

(71) Applicant: Arizona Board of Regents on Behalf of The University of Arizona, Tucson, AZ (US)

(72) Inventor: Jeong-Yeol Yoon, Tucson, AZ (US)

(21) Appl. No.: 18/253,905

(22) PCT Filed: Nov. 22, 2021
(86) PCT No.: PCT/US21/60355

§ 371 Date: May 22, 2023

Related U.S. Application Data
(60) Provisional application No. 63/116,747, filed on Nov. 20, 2020.

(51) Int. Cl. G01N 21/64; G01N 33/543; G01N 33/569

(52) U.S. Cl. CPC G01N 21/6428; G01N 21/6458; G01N 33/54389; G01N 33/56983; G01N 2021/6439; G01N 2333/165

(57) ABSTRACT

A device for detecting and/or quantifying a coronavirus comprising a paper microfluidic chip, and the use thereof.

### CROSS-REFERENCE TO RELATED APPLICATIONS

This application claims the benefit of Provisional Appl. No. 63/116,747, filed Nov. 20, 2020. The content of the aforesaid applications are relied upon and are incorporated by reference herein in their entirety.

### FIELD OF THE INVENTION

The field of the invention relates generally the measurement and/or detection of viruses.

### BACKGROUND

There is a strong need to develop a handheld, rapid, and extremely sensitive assay of coronaviruses such as SARS-CoV-2 amid the recent COVID-19 pandemic.

The gold standard test for diagnosing viruses such as SARS-CoV-2 is a molecular test called reverse transcription quantitative polymerase chain reaction (RT-qPCR), which can be developed quickly after discovering a new virus. It is highly accurate as it relies on detection of amplified genetic information. However, RT-qPCR requires complex laboratory equipment and is expensive to implement. In high-volume scenarios such as in the middle of an active pandemic, it may force patients to wait for a long time before receiving results. In contrast, point-of-care biosensors that rely on binding between antigens and antibodies allow for inexpensive and rapid large-scale testing, as the equipment is cheap and the results can be obtained quickly. However, these tests often suffer from lower accuracy and sensitivity compared to molecular tests. Truly effective screening requires tests that are both accurate as well as inexpensive and easy to deploy on a massive scale, so that even crowded settings or resource-limited environments can efficiently screen all members of a population. Such tests could even be administered at home, so that individuals with mild cases of illness can repeatedly test themselves in order to know when they are safe to return to public settings.

As the immunoassays are not sensitive and have high limit of detection (high LOD), early-stage infections cannot be identified; SARS-CoV-2 is notoriously known for its early-stage propagation. In addition, they also suffer from false-positive results when used with complicated samples (NP or other swabs).

The COVID-19 pandemic has exposed our society's need for rapid, low-cost diagnostic tests that are highly sensitive. While genetic amplification and culturing techniques can accurately detect SARS-CoV-2, they require expensive laboratory equipment and may have long processing times during high-volume stages of the pandemic. Alternatively, point-of-care (POC) immunoassays are inexpensive and user friendly, but they often have inferior sensitivity. All these existing methods require uncomfortable nasopharyngeal (NP) or anterior nasal swabs, which may expose healthcare staff to the virus. The inventor has developed a smartphone fluorescence microscope-based immunofluorescence particle counting assay to detect SARS-CoV-2 down to the limit of detection of 10 ag/μL. The invention is affordable, more sensitive than similar rapid kits, and works with saline gargle samples, which are comfortably and easily obtained from the patient and may pose less risk to healthcare staff during sample collection.

### DESCRIPTION

### Definitions

For the purposes of promoting an understanding of the principles of the invention, reference will now be made to certain embodiments and specific language will be used to describe the same. It will nevertheless be understood that no limitation of the scope of the invention is thereby intended, and alterations and modifications in the illustrated invention, and further applications of the principles of the invention as illustrated therein are herein contemplated as would normally occur to one skilled in the art to which the invention relates.

Unless defined otherwise, all technical and scientific terms used herein have the same meaning as commonly understood by one of ordinary skill in the art to which this invention pertains.

For the purpose of interpreting this specification, the following definitions will apply and whenever appropriate, terms used in the singular will also include the plural and vice versa. In the event that any definition set forth below conflicts with the usage of that word in any other document, including any document incorporated herein by reference, the definition set forth below shall always control for purposes of interpreting this specification and its associated claims unless a contrary meaning is clearly intended (for example in the document where the term is originally used).

The use of “or” means “and/or” unless stated otherwise.

The use of “a” or “an” herein means “one or more” unless stated otherwise or where the use of “one or more” is clearly inappropriate.

The use of “comprise,” “comprises,” “comprising,” “include,” “includes,” and “including” are interchangeable and not intended to be limiting. Furthermore, where the description of one or more embodiments uses the term “comprising,” those skilled in the art would understand that, in some specific instances, the embodiment or embodiments can be alternatively described using the language “consisting essentially of” and/or “consisting of.”

As used herein, the term “about” refers to a ±10% variation from the nominal value. It is to be understood that such a variation is always included in any given value provided herein, whether or not it is specifically referred to.

As used herein, the term “antibody” refers to an antibody to the virus (e.g., an enteric virus) being detected. For example, Rabbit polyclonal antibody to norovirus capsid protein VP1 (anti-norovirus) may be used as an antibody in the detection of norovirus.

As used herein, the term “fluorescent particle” refers to a polymeric particle (e.g., a nanoparticle or microparticle) attached a fluorescent dye, such as a fluorescent polystyrene particle, more preferably carboxylated, yellow-green fluorescent, polystyrene particles. For example, fluorescent particle may have a diameter in the range of about to 0.2 μm to about 2 μm (or about to 0.2 μm to about 1 μm; or about to 1 μm to about 2 μm; or about to 0.4 μm to about 0.8 μm; or about to 0.4 μm to about 0.6 μm; or 0.2 μm to about 0.7 μm). In some embodiments, the diameter may be 0.5 μm.

As used herein, the term “smartphone-based fluorescence microscope” refers to a fluorescence microscope involving the use of a smartphone. In some embodiments, the term, “smartphone-based fluorescence microscope” encompasses a microscope attachment to a smartphone, modified with two light emitting diodes (LED's) and two color filter films.

As used herein, the term “smartphone” refers to a portable device with digital cameras and computing functions. In some embodiments, the term “smartphone” encompasses a portable device with computing functions and mobile telephone functions into one unit.

### BRIEF DESCRIPTION OF FIGURES

FIG. 1. Outline of an exemplary diagnostic process. Saline gargle sample is collected, loaded onto the paper-based microfluidic chip. Antibody conjugated fluorescent particles are then loaded and flowed through the paper-based microfluidic channels passively via capillary action. This led to particle immunoagglutination, whose extents are imaged with a smartphone-based fluorescence microscope. Only the immunoagglutinated particles are isolated and their pixel sums are counted from the multiple images on a single channel. Using a threshold pixel count, image processing leads to determination of a positive or negative result. Using clinical samples, this assay achieved 100% sensitivity and 93% accuracy, with Ct values ranging from 26 to 35. Portions of this figure were created using BioRender.com.

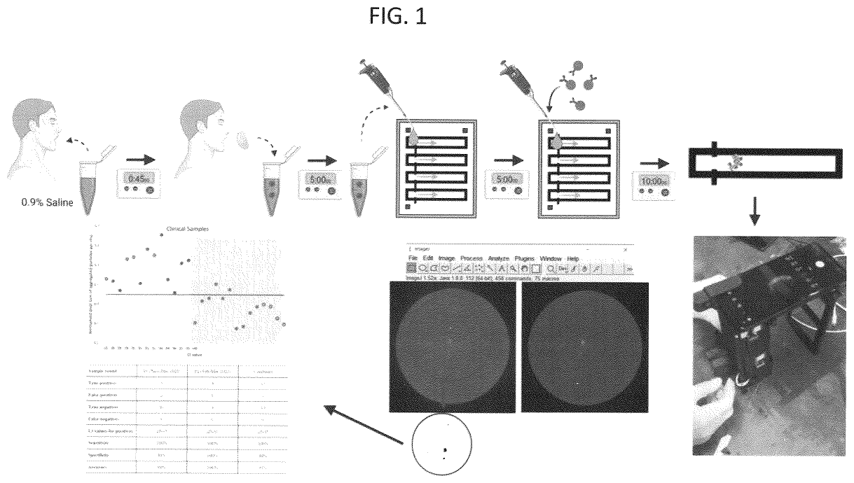

FIG. 2. Smartphone-based fluorescence microscope for imaging particle immunoagglutination on a paper-based microfluidic chip. (A) SolidWorks diagram of smartphone-based fluorescence microscope device. (B) Parts assembly of smartphone-based fluorescence microscope. (C) Foldable smartphone stand for portability. (D) Paper-based microfluidic chip design and chip holder. (E) Excitation light source and emission spectrum. (F) Circuit diagram and the circuit photograph for the light source. (G) Final device demonstration. (H) Optical system diagram. (I) Comparison between the fluorescent particle images of the paper-based microfluidic chip from a conventional fluorescence microscope and from the developed smartphone device, both at the same location. The processed image from the developed smartphone device is also shown.

FIG. 3. Image processing procedure. (A) Photograph of human clinical saline gargle samples: positive (left), negative (right), and deionized (DI) water in the center for comparing turbidity. (B) After loading the positive sample and the antibody-conjugated fluorescent particles to a microfluidic channel, the channel is imaged and the images are opened in ImageJ, (C) converted to 8-bit grayscale, and (D) the threshold is adjusted using the histogram tool until (E) only the particles are shown in white. (F) Particle areas for all 16 images (4 images/channel×4 channels) are saved in Microsoft Excel, and particles with areas below 30 or above 10,000 pixels are excluded before summing the particles in each image and obtaining the average summed particle area per image (highlighted with arrow). (G-J) The same process is repeated for the negative sample.

FIG. 4. Assay optimizations. Effects of various parameters on the ability to distinguish between negative and positive samples, including: (A)-(B) drying time (n=3), (C)-(D) antibody type (n=1), (E)-(F) order of sample and particle loading (n=3), and (G)-(H) addition of Tween 20 to the antibody-conjugated particle preparation (n=1). All further assays were conducted with immediate imaging, polyclonal antibodies, loading samples before loading antibody-particle suspensions, and adding Tween 20 to the particle suspensions.

FIG. 5. LOD and specificity using simulated saline gargle samples (10% saliva and 0.9% saline). (A) A standard curve was created including multiple concentrations of SARS-CoV-2 spiked into saline gargle samples (n=3 for each concentration, each time using a different chip). (B) Specificity was tested using negative simulated saline gargle samples, positive SARS-CoV-2 (1 pg/μL) simulated saline gargle samples, and influenza A/H1N1 (Ct value=25-28) simulated saline gargle samples (n=3 for each sample, each time using different chip).

FIG. 6. Assay results with human clinical samples. Saline gargle samples included high-positive SARS-CoV-2 (Ct=25) to low-positive SARS-CoV-2 (Ct=35), as well as negative samples (Ct>40, shaded in orange). Solid blue and orange n=19 samples were tested in round #1 without any sedimentation steps, and patterned n=8 samples were tested in round #2 with sedimentation. All pixel sums are normalized by the cutoff values, creating the cutoff line of 1. The cutoff pixel sum is 1700 for all round #1 samples and 500 for all round #2 samples.

FIG. 7. Outline of an exemplary protocol. a) 4 μL of sample is added to the center of each channel of the chip. b) 2 μL of antibody-conjugated fluorescent particle suspension is loaded to the center of each channel of the chip. c) These particles flow via capillary action and aggregate with the norovirus captured on the chip. d) These chips can comprise a single channel or may contain four channels per chip.

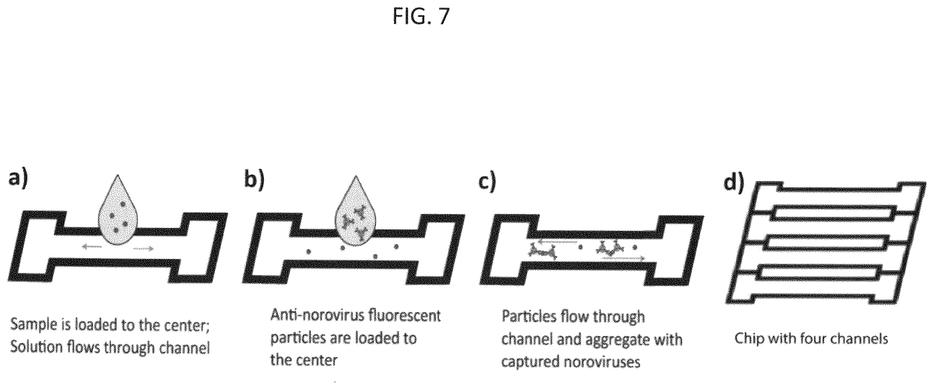

Ever since the onset of the pandemic, emphasis was placed on development of rapid antigen assays specific for SARS-CoV-2 as a replacement for the gold standard method of RT-qPCR4. A common factor in many of these rapid tests is the way that the sample is collected, which is via nasopharyngeal (NP) or anterior nasal swab. Several such tests are already commercially available to patients, such as Abbott's BinaxNOW™ COVID-19 antigen card. However, even this test suffers from low sensitivity in certain populations, and the nasal swab may be uncomfortable to patients and may increase the risk of infection for staff or caregivers. Very recently, saliva specimens have drawn interest as a potential sample collection method due to their ease of collection, speed, low cost, and decreased risk to healthcare providers. Saliva is an integrated mixture of secretions that contains a large number of proteins including immunoglobulins, enzymes, metabolites, hormones, and electrolytes. This makes the medium an attractive option for detecting pathogens and quantifying biomarkers that can provide relevant information about the immunological, inflammatory, endocrine, and metabolic status of patients. The main challenge presented by this testing method is the quality of patient samples. Samples may require dilution or pretreatment due viscosity, and antigens may be more difficult to detect in this specimen type. Depending on whether or not a patient has consumed food or beverages (or used toothpaste) prior to collection of a saliva sample, there may be significant contaminants present that may hinder the performance of immunoassays and nucleic acid amplification detection methods. The inventor has successfully developed and implemented a saline gargle sample collection method. Individuals were given 5 mL of 0.9% sterile saline and completed 3 cycles of a 5 second swish followed by a 10 second gargle. Positive samples were confirmed by RT-qPCR using CDC RUO primers and protocol.

Enteric viruses such as norovirus can be detected at the single copy level by testing water samples using a paper microfluidic chip and a smartphone-based fluorescence microscope. Antibody-conjugated, fluorescent particles will be loaded onto the paper microfluidic chips and react with target antigens in the sample to form immunoagglutination. Immunoagglutinated particles will be counted one-by-one through spreading them over the length of microfluidic channel via capillary action using a smartphone-based fluorescence microscope.

The invention encompasses detecting coronaviruses such as SARS-CoV-2, at the single copy level in saliva (buccal swab) samples and surface swabs (e.g., nasopharyngeal (NP) swab).

For example, invention encompasses a device that is handheld, 2) provides rapid results (e.g., <10 min from sample-to-answer), and 3) automated assay. In some embodiments, the assay involves the collection of signals with a smartphone and where results are transmitted to the central cloud storage. These data can then be utilized to increase the accuracy of e.g., SARS-CoV-2 exposure assessments and aid in decision-making for better disease tracking and resource allocation.

One aspect of the invention pertains to a smartphone-based antigen assay device.

In some embodiments, smartphone-based antigen assay device comprises a fluorescence microscope with at least there components/three modules (i.e., optical, housing, and electrical).

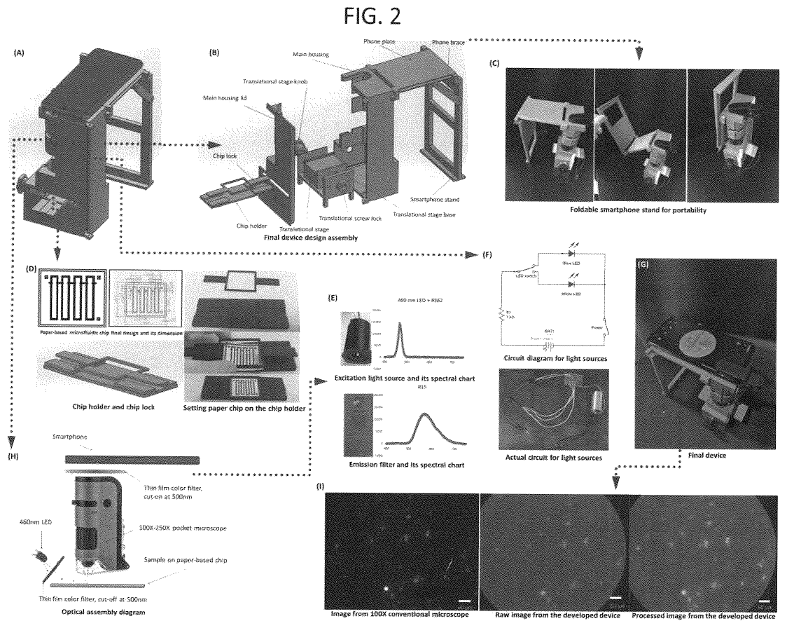

In certain embodiments, the device encompasses a smartphone-based fluorescence microscope with an optical module comprising a higher-quality microscope attachment providing 100×-250× magnification (e.g., B07NW5Z3WF; Carson Optical, Ronkonkoma, NY, USA). The microscope may include a clip that is compatible a smartphone (see e.g., FIG. 2H). The microscope may also include thin filter films such as color filter films (e.g., Color Filter Booklet, Edmund Optics, AZ, USA) to achieve excitation light (cut-off before 500 nm) and emission light (cut-on after 500 nm). A spectrophotometer (e.g., Ocean Optics, Inc, Dunedin, FL, USA) may be used to determine the output of the LED-filter combinations (see e.g., FIG. 2E). Three LEDs (wavelengths of 405, 460, and 490 nm) combined with the selected color filter films were observed for the set that provided ideal contrast between fluorescence signal and background paper autofluorescence.

In some embodiments, for the housing module, a translational stage system may be added to provide smooth and precise movement along both x- and y-axes (e.g., using a T8 lead screw (see e.g., the design in FIG. 2B)). Furthermore, a foldable phone stand may be included to achieve both steadiness of the phone while the device is in use, as well as ease of transport when moving the device (see e.g., FIG. 2C).

In some embodiments, for the electrical module, two light sources, including illumination light (white light for overall chip observation) and excitation light (for generating the fluorescence signal) may be installed and may be powered by one or more batteries (e.g., 9 V rechargeable battery) (FIG. 2F).

An exemplary device of the invention is shown in FIG. 2G.

After using image processing, the fluorescence imaging results are comparable to those of a benchtop fluorescent microscope (Nikon Eclipse TS100, Minato, Tokyo, Japan) with ISCapture software using blue excitation and green emission wavelength filter attachments (A.G. Heinze, Lake Forest, CA, USA) (FIG. 2I). The total cost of an exemplary device according to the invention, which can interface with a user's own smartphone, is less than $50.

Another aspect of the invention pertains to a wholly novel technology of using a smartphone-based microscope and particle counting on a paper microfluidic chip coronaviruses at the single copy level. The antibody-conjugated fluorescent polystyrene particles are pre-loaded onto the paper microfluidic chip. Upon loading the sample, cells and tissue fragments are filtered by the paper fibers, and the target antigens flow through the paper pores via capillary action and induces the particles to aggregate. A smartphone-based fluorescence microscope can count such aggregation one-by-one, as the concentration of these particles is extremely low, and they spread over the length of the microchannel to facilitate such particle-by-particle counting.

To accommodate coronavirus assay from nasal, nasopharyngeal (NP), or buccal swabs, as well as saliva samples, following the device may include modifications such as:

Pore size. Pore size of microfluidic paper (e.g., nitrocellulose paper) used to make paper microfluidic chips may be adjusted. For example, larger pore sizes, e.g. 5 μm to 15 μm, may be used.

Channel Width. With the enlarged pore size, the capillary flow will be increased. To compensate this change, wider channel width may be used (e.g., 2.4 mm up to 5 mm) and/or longer channel length (e.g., 21 mm up to 50 mm) may be used.

Changes in pore size and channel dimension, along with the presence of nasal or saliva components, will necessitate the adjustments of 1) volume and concentration of antibody conjugated particles that is loaded onto the paper microfluidic chip (currently 2 uL at 0.01-0.02%; volume can be adjusted from 2 uL to 6 uL and the concentration from 0.001% to 0.04%), 2) volume of target sample (currently 4 uL; can be adjusted from 4 uL to 8 uL).

Filter Card. Acrylic films (filter cards) can be used to further optimize the excitation and emission wavelengths, as some components in the above samples (nasal or saliva samples) may auto-fluoresce.

Detection. For smartphone detection, the intensity threshold may be mean+30 to mean+70. Size threshold may be from mean+10 to mean+50.

In other embodiments, wherein the device of the invention comprises a smartphone-based fluorescence microscope comprising a smartphone, a microscope attachment, a light source (such as LED), a battery to power said light source (such as LED) (e.g., a button battery), and an optical filter.

Without being limited by theory, in some embodiments, the device may involve the use of a cover slip, which is added to “flatten” the paper chip as some types of paper such as nitrocellulose paper tends to become curled occasionally.

The paper microfluidic chip may also be simply placed on a glass slide and placed into the device for smartphone fluorescence imaging.

In further embodiments, the device may include a microscope attachment, an LED, a battery to power LED (e.g., a button battery), and an optical filter are housed within an enclosure to block ambient lighting. The enclosure may comprise plastic and/or metal (e.g., a plastic enclosure).

In some embodiments, multiple images may be taken from a single channel of a paper microfluidic chip, the exact positions of these images can be randomly chosen. For instance, four images may be taken from a single channel of a paper microfluidic chip.

Exact positions of these four images can be randomly chosen. In the further embodiments of the device, the slide that accommodates the paper chip has multiple “stops” to position the paper chip at multiple different fixed locations. This will make the user to position the chip in an easier and reproducible manner.

The microscope attachment of the device may comprise a bandpass filter or acrylic films (also known as “filter cards”).

One aspect of the invention pertains to a device for detecting and/or quantifying a coronavirus (e.g., SARS-CoV-2 virus) comprising a paper microfluidic chip, comprising one or more microfluidic channels, wherein said paper has a pore size of about 5 μm to 15 μm, and wherein said channels have a width about 2 mm to about 5 mm and/or a channel length between channel length of about 20 mm to about 50 mm. In some embodiments, the device comprises a benchtop fluorescence microscope. In certain embodiments, the device further comprises a smartphone-based fluorescence microscope comprising a smartphone, a microscope attachment, a light source (such as LED), a battery to power said light source (such as LED) (e.g., a button battery), and an optical filter. In some embodiments, the device further comprises a microscope attachment, an LED, a battery to power LED (e.g., a button battery), and an optical filter are housed within a plastic enclosure to block ambient lighting. The microscope attachment may include a bandpass filter or acrylic films (also known as “filter cards”).

Another aspect of the invention pertains to a method for detecting a coronavirus, said method comprising
(a) applying a suspension comprising said virus (e.g., SARS-CoV-2 virus) to a paper microfluidic chip;
(b) adding an anti-virus antibody conjugated fluorescent particle suspension to the paper microfluidic chip;
(c) allowing particles and viruses spread spontaneously throughout the paper microfluidic channel via capillary action, allowing the particles to aggregate and facilitating imaging of individual particles.

In some embodiments, the virus is present in a concentration ranging from 10⁰ to 10⁵ virions, or a concentration ranging from 10⁰ to 102 virions. In further embodiments, the method involves taking said measurement without using any sample concentration or nucleic acid amplification step.

In some embodiments, said paper microfluidic chip comprises nitrocellulose paper, cellulose paper, or polymeric fiber filter.

In some embodiments, wherein said suspension has not been pre-purified, pre-concentrated, or pre-amplified prior to testing.

In some embodiments, said method involves a single virus copy level detection of said virus.

In some embodiments, said imaging and counting aggregation of antibody-conjugated, fluorescent submicron particles is on-paper.

In some embodiments, said antibody is a polyclonal antibody or a monoclonal antibody.

In some embodiments, the fluorescent particle is a fluorescent polystyrene particle.

In some embodiments, said method further comprises:
(1) fabricating a paper microfluidic chip with multiple channels on it for simultaneously conducting multiple assays;
(2) conjugating an antibody to fluorescent particles to obtain an anti-virus antibody conjugated fluorescent submicron particle suspension;
wherein said steps are performed prior to said steps (a)-(c).

In some embodiments, said method further comprises:
(i) imaging the aggregation of antibody conjugated fluorescent particles;
(ii) removing the background noises and autofluorescence from paper substrate using an optimized threshold intensity and isolating only the fluorescent particles;
(iii) binarizing an entire image;
(iv) removing the smaller size of particles to isolate only the aggregated particles;
(v) relating the total pixel area to the virus concentration to construct a standard curve and estimate the virus concentration from an unknown sample;
wherein said steps are performed after said steps (a)-(c).

Another aspect of the invention pertains to a kit for detecting a virus (such as an enteric virus) comprising
a paper microfluidic chip with a pore size of about 5 μm to 15 μm depending on the type of sample suspension,
a suspension of antibody conjugated fluorescent particles (wherein the volume of the antibody conjugated fluorescent particles is from 2 uL to 6 uL and a concentration from about 0.001% to about 0.04%, depending on the type of sample suspension), and optionally a smartphone-based fluorescence microscope.

A further aspect of the invention pertains to kit for detecting a virus (such as an enteric virus) (e.g., coronavirus) comprising
(a) a device of the invention; and
(b) one or more reagents (e.g., fluorescent particles conjugated with different antibodies) for carrying out detection and/or quantification of one or more coronaviruses.

A further aspect of the invention pertains to a method for detecting a virus (such as an enteric virus) comprising
(a) fabricating a paper microfluidic chip with multiple channels on it for simultaneously conducting multiple assays;
(b) conjugating an antibody to fluorescent particles to obtain an anti-virus conjugated fluorescent submicron particle suspension;
(c) applying a suspension comprising said virus to the paper microfluidic chip;
(d) adding said antibody conjugated fluorescent particle suspension to the paper microfluidic chip;
(e) allowing particles and viruses spread spontaneously throughout the paper microfluidic channel via capillary action, allowing the particles to aggregate and facilitating imaging of individual particles;
(f) imaging the aggregation of antibody conjugated fluorescent particles;
(g) removing the background noises and autofluorescence from paper substrate using an optimized threshold intensity and isolating only the fluorescent particles;
(h) binarizing an entire image;
(i) removing the smaller size of particles to isolate only the aggregated particles;
(j) relating the total pixel area to the virus concentration to construct a standard curve and estimate the virus concentration from an unknown sample.

### EXAMPLES

Image Processing Procedure

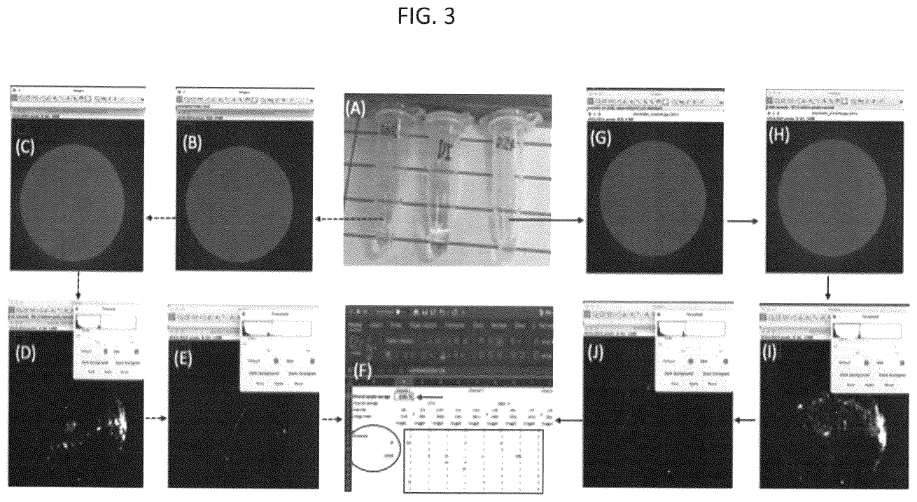

After the paper-based microfluidic assay was completed for an entire chip (set of 4 channels), images were collected using a smartphone (Galaxy S20; Samsung Electronics America, CA, USA) and the fluorescence microscope attachment shown in FIG. 2. Using the Pro setting on the Samsung camera app, we collected four images per channel, starting from the loading pad and moving the field of view (FOV) towards the flow front. Key camera settings were an ISO value of 50, an exposure time of 0.5 seconds, a white balance value of 4000K, and manual focus enabled. Images were analyzed using ImageJ, an open-source software (U.S. National Institutes of Health, Bethesda, MD, USA). This process was expedited with the use of macros which can be recorded and set in ImageJ. FIG. 3 represents the steps of the process for one positive sample (FIGS. 3B through 3E) and one negative sample (FIGS. 3G through 3J). All raw images collected from testing the samples were uploaded to a laptop computer. One at a time, each image was imported into ImageJ individually and converted to 8-bit grayscale. Images were subsequently thresholded to isolate the fluorescence of the antibody-conjugated fluorescent particles using a thresholding tool built into ImageJ. Our method utilized the default setting, to make a binary distinction between fluorescing particles and background. Threshold values were determined manually for each individual image, due to variation in overall brightness of each image. After thresholding was complete, for most images the target aggregates represented 0.01% of the total pixel area of each image. FIGS. 3D and 31 show thresholded images where some fluorescence signal and background intensity are still visible. FIG. 3E shows the final masked image for a positive sample, with two large particles particularly noticeable. FIG. 3J shows the same for a negative sample where no large aggregates are noticeable. After thresholding, the “Analyze Particles” function in ImageJ was used to collect all particle areas, which were denoted by pixel values of 255 (white) compared to the background pixel values of 0 (black). This data was then exported into Microsoft Excel (FIG. 3F) where an additional minimum size threshold of 30 pixels was used to eliminate any remaining noise or non-aggregated singlet particles leftover after the thresholding. For example, all the cells in FIG. 3F with a pixel count of 1 would be eliminated using this minimum size thresholding step. A maximum size threshold of 10,000 was used to eliminate particles that were extremely large, typically only occurring occasionally. If the paper chip was dirty and many large clumps were formed, the entire channel was eliminated.

Assay Optimizations

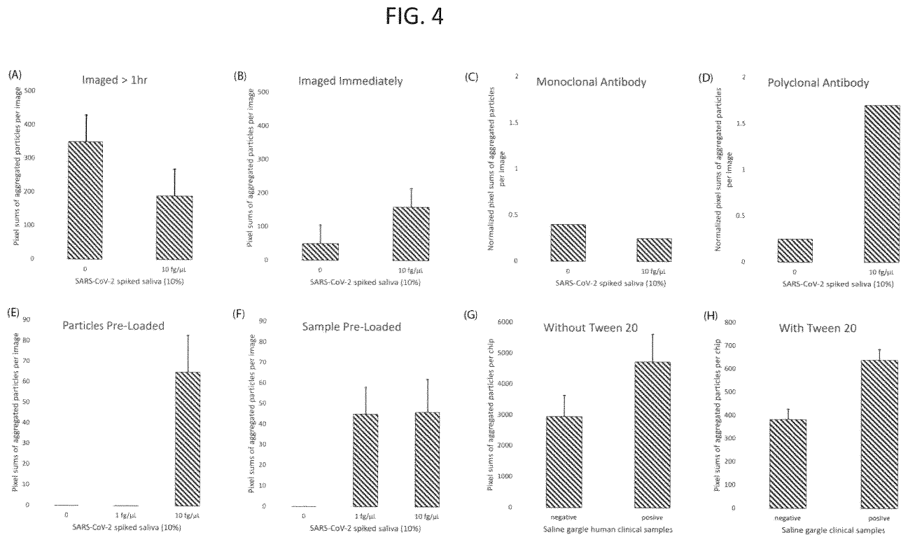

There were multiple variables that influenced the quality and reliability of results using the immunofluorescence particle counting assay. Drying time was found to have a significant influence not only on the separation between positive and negative samples, but also on the general amplitude of signal (pixel counts of immunoagglutinated particles) collected. Based on our observations, longer drying time was associated with larger particle clumps. This occurred in all samples and increased standard error (FIG. 4A). To reduce variation from this effect, we chose to make immediate imaging of the chips an element of the final protocol, as demonstrated by the results in FIG. 4B.

We also optimized the type of antibody used for our assay. SARS-CoV-2 detection from saliva samples is less sensitive and presumably even less with saliva gargle samples (due to the dilution by saline solution), compared to nasopharyngeal swabs 12,14. Typical viral loads in saliva vary from 104 to 108 copies/mL, equivalent to 101 to 105 copies/μL (=10 fg/μL to 100 pg/μL, considering one copy is approximately 1 fg), during the first week of symptoms 15. Although monoclonal antibodies are more specific to their target antigen, we found better differentiation between positive and negative samples using polyclonal nucleocapsid antibodies at 101 copies/μL (=10 fg/μL), which is within our target range of concentrations (see FIGS. 4C and 4D). The difference between spike and nucleocapsid targeting antibodies was not tested in this assay optimization.

The order of loading solutions (antibody-conjugated fluorescent particles first, or clinical sample first) was also found to have an influence on the results of the assay. Although both methods showed a difference between negative and positive (10 fg/μL) samples (FIGS. 4E and 4F, respectively), pre-loading the sample first allowed for detection at a lower viral load.

Possibly the greatest improvement to the assay was the addition of Tween 20 surfactant to the preparation protocol of the antibody-conjugated fluorescent particles. This was tested using clinical saline gargle samples with both negative and positive SARS-CoV-2 diagnoses. Without Tween 20 added to the particles, there was cross-over between negative and positive targets (FIG. 4G). Once the assay was repeated using the same samples but with Tween 20 added to the particle preparation, a clear difference between the negative and positive samples emerged (FIG. 4H).

Subsequent to assay optimizations, all further assays, which correspond to the results demonstrated in this paper, were conducted with immediate imaging, polyclonal antibodies, loading samples before loading antibody-particle suspensions, and adding Tween 20 to the antibody-conjugated particle suspensions.

Limit of Detection and Specificity Using Simulated Saline Gargle Samples

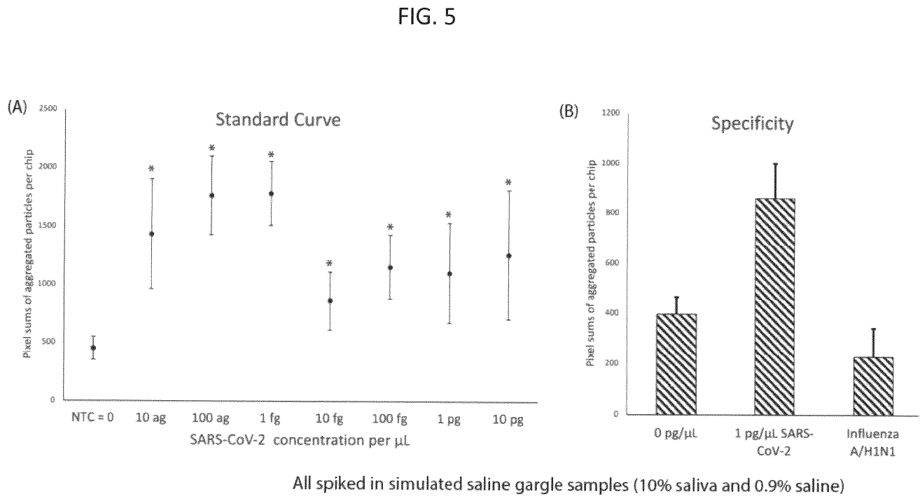

Simulated saline gargle samples were prepared using pooled human saliva (IRHUSL250ML; Innovative Research; Novi, MI) and UV-inactivated SARS-CoV-2 (WA/2019, WRCEVA) in 5% DMEM media in serial dilutions. The concentration of the SARS-CoV-2 stock was 1.7×107 PFU/mL, which is estimated to correspond to 10 pg/μL. Experiments were repeated with varying concentration of the spiked SARS-CoV-2, while maintaining identical saliva dilution (10%=approximated saliva content in saline gargle samples) and saline content (0.9%=used for saline gargle sampling). FIG. 5A shows the LOD of 10 ag/μL using simulated saline gargle samples. Since the mass of a single SARS-CoV-2 virus is estimated at 1 femtogram 16 and the sample volume per channel is 4 μL, this LOD corresponds to 40 ag or 0.04 copy per channel. This low LOD is theoretically possible, as the assay detects antigens rather than the intact viruses. The results show a hook effect, initial increase, peaking at 1 fg/μL, and decrease at high concentrations, typical of immunoassays. All concentrations maintain significance with p<0.05.

An additional test was performed to evaluate specificity of the assay when the solution was spiked with influenza A/H1N1 (NATFLUAH1-ERCM; ZeptoMetrix, Buffalo, NY). There was no significant difference between negative simulated samples and influenza A/H1N1 (Ct value=25-28) samples, while a significant difference was found between influenza A/H1N1 samples and positive (1 pg/μL) SARS-CoV-2 samples (p<0.05) (FIG. 5B).

Assay Results with Human Clinical Samples

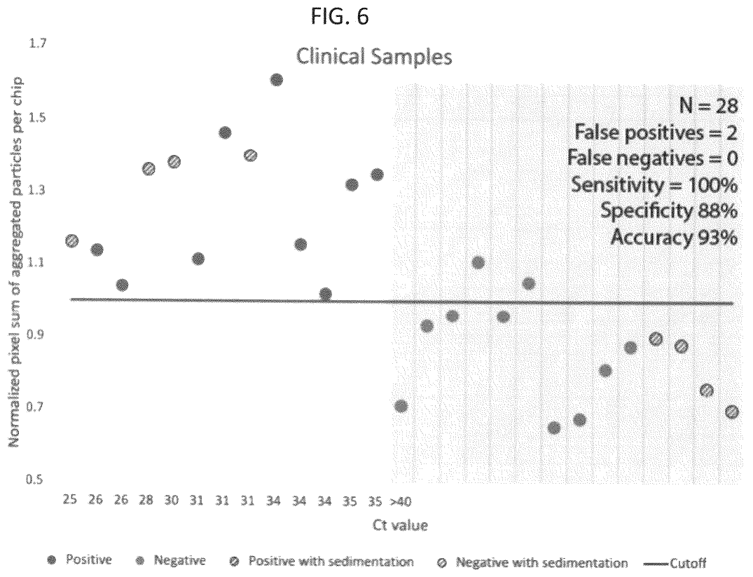

Each data point in FIG. 6 represents a sample from an individual patient, assayed on one chip with four channels, thus replicated by four times. All images were analyzed to acquire total pixel sums of particle immunoagglutination. FIG. 6 shows the clinical saline gargle results from all clinical samples collected in October 2020. Samples were delivered in two rounds, one in December of 2020 (round #1) and the other in March 2021 (round #2). The first round of samples was pre-selected for quality and minimal contamination via visual inspection, whereas the second round was chosen at random. The samples received in March of 2021, although collected with the same procedure and at the same time as the first round, exhibited significantly higher turbidity and colorations, possibly due to contamination from food or oral hygiene products (i.e., toothpaste). Due to higher turbidity and coloration, round #2 samples were sedimented using a mini-centrifuge (dual rotor personal microcentrifuge; item #2461-0016, USA Scientific, Ocala, FL) at 6000 RPM for 10 seconds, and only the supernatant was loaded onto the paper-based microfluidic channel, in order to help eliminate addition of any food or large particles to the paper. This sedimentation resulted in a loss of virus and saliva samples, resulting in different pixel sum values. Hence, separate threshold decision boundaries were generated for each data set, and data points were normalized to their respective threshold values to generate a single plot. FIG. 6 shows the clinical saline gargle sample results of both round #1 and round #2 combined. These samples showed clear separation with positive target samples (Ct<35) and negative samples (Ct>40), as confirmed by RT-qPCR. RNA was extracted from samples and then quantified by a reverse transcription polymerase chain reaction (RT-qPCR) assay. Following CDC guidelines and protocols, the SARS-CoV-2 (2019-nCoV) CDC RUO primers were used to determine the presence of SARS-CoV-2 in samples. For the positive samples, a hook effect can also be observed against Ct values.

There were no requirements in sample collection with regard to last oral intake (LOI). And there was no clear correlation between the LOI and the normalized pixel sums. However, when LOI was 30 minutes or less, the saline gargle method produced false negatives. Saline gargle samples were collected with paired nasopharyngeal swabs which were not impacted by LOI, but had a decreased sensitivity compared to saline gargle. It is possible that an individual with an LOI<30 minutes would generate a false negative in both sample types.

A sensitivity of 100% was achieved for all Ct values from both rounds, and the level of turbidity of the samples did not seem to affect the sensitivity of results, although the cutoff pixel sum values were different for two rounds of assays. Specificity [true negative/(true negative+false positive)] and accuracy [(true positive+true negative)/(true positive+true negative+false positive+false negative)] of round #1 samples were 83% and 90%, respectively, indicating that only two negative samples out of 19 were identified positive. We cannot rule out the possibility of these samples being positive, potentially with very low viral load (since our LOD is very low), which RT-qPCR failed to amplify. Meanwhile, specificity and accuracy of round #2 were 100% and 100%, respectively, despite being more turbid in general. With both rounds combined, the overall specificity was 100%, overall specificity was 88%, and overall accuracy was 93%. Assay performance of each round is summarized in Table 2.

TABLE 2
Assay results with clinical samples.

| Sample round | #1 (December 2020) | #2 (March 2021) | Combined |
|---|---|---|---|
| True positives | 9 | 4 | 13 |
| False positives | 2 | 0 | 2 |
| True negatives | 10 | 4 | 14 |
| False negatives | 0 | 0 | 0 |
| Ct values for | 25-35 | 25-31 | 25-35 |
| positives | | | |
| Sensitivity | 100% | 100% | 100% |
| Specificity | 83% | 100% | 88% |
| Accuracy | 90% | 100% | 93% |

Assay Performance

A fully optimized assay for SARS-CoV-2 detection from simulated and clinical saline gargle samples was realized. The LOD of 10 ag/μL (or 40 ag/assay) was achieved with simulated saline gargle samples and Ct=35 (typical upper limit of RT-qPCR assays) with clinical saline gargle samples. The multiple iterations of optimizing preparation, loading, and drying time of the assay (FIG. 4) led to the final protocol of pre-loading sample, then loading polyclonal antibody-conjugated fluorescent particles diluted with Tween 20, and imaging the chips shortly afterwards. In comparison to our group's earlier publications, which typically have used one of four channels on the chip per test 13,17, we here represent the average particle sum of all four channels on the chip as a single data point in FIGS. 5 and 6. This was done to mitigate effects of variation in paper type, wax printing, and particle concentration per channel.

One concern with using polyclonal antibodies was the effect on specificity of the assay. FIG. 5B shows that specificity is maintained when tested against influenza A/H1N1 virus. The presence of H1N1 in the simulated saline gargle sample had a lower signal than even the no target control (no virus in the simulated saline gargle sample), possibly indicating that the influenza A/H1N1 virus reduced any non-specific aggregation that can occur with no target control samples. The hook effect seen in FIG. 5A is an expected occurrence in immunoassays 18. The signal peaks between 100 ag/μL and 1 fg/μL, with the higher concentrations producing lower signals. This is called the post-zone effect and is most likely occurring due to the high amount of antigen over-saturating the number of antibodies available, resulting in a decrease in the extent of particle immunoagglutination. Regardless of the lower signal, the higher concentrations of SARS-CoV-2 tested were still significantly different from the no target control sample (p<0.05).

Evaluation of Clinical Samples

Samples were collected in October 2020 and then received for testing in two separate batches, the first round in December of 2020 and the second in March of 2021. Our assay demonstrated comparable performance in distinguishing positive and negative SARS-CoV-2 clinical samples. Somewhat similar to the hooked trend in FIG. 5A, there is a peak signal and post-zone effect visible in FIG. 6. For the first round of samples (FIG. 6), a high signal was achieved with the lowest concentrations of SARS-CoV-2 (Ct=34-35) and the peak signal occurred at Ct=30-31. The highest concentrations (Ct=25-26) in the post-zone area had the lowest signal. We demonstrated 100% sensitivity for both rounds of samples, but the two false positives from the first round reduced the specificity and accuracy to 88% and 93%, respectively. The cutoff value for both datasets was manually chosen to have the highest separation between negative and positive, but it was noticed that the ideal cutoff range is roughly equal to the average y-axis value of all data points on the graph. The successful distinction between positive and negative saline gargle samples is promising and further testing should be done to more thoroughly assess our method's potential for reliable diagnostics.

The samples from the second round of testing appeared to be more viscous, turbid, and contaminated with food and drink particulate. To resolve this issue, we incorporated an additional sedimentation step to eliminate food particulates. Despite the effect of last oral intake (LOI) appears to have on other assays, our method consistently demonstrated strong sensitivity, specificity, and accuracy, all 100%, as seen in Table 2. We can expect that the addition of sedimentation step to the first-round samples would improve the sensitivity, specificity, and accuracy close to 100%, although we were not able to do so due to the sample availability and the approved IRB protocol.

This method would be particularly useful in resource-limited areas where testing methods that require purification and laboratory settings may not be available.

1. A device for detecting and/or quantifying a coronavirus comprising a paper microfluidic chip, comprising one or more microfluidic channels, wherein said paper has a pore size of about 5 μm to 15 μm, and wherein said channels have a width about 2 mm to about 5 mm and/or a channel length between channel length of about 20 mm to about 50 mm.

2. The device of claim 1, wherein said virus is a SARS-CoV-2 virus.

3. The device of claim 1, wherein said device further comprises a benchtop fluorescence microscope.

4. The device of claim 1, wherein said device further comprises a smartphone-based fluorescence microscope comprising a smartphone, a microscope attachment, a light source, a battery to power said light source, and an optical filter.

5. The device of claim 4, wherein said microscope attachment, LED, battery to power LED, and optical filter are housed within a plastic enclosure to block ambient lighting.

6. The device of claim 4, wherein said microscope attachment comprises a bandpass filter or acrylic films.

7. A method for detecting a virus, said method comprising
(a) applying a suspension comprising said virus to a paper microfluidic chip;
(b) adding an anti-virus antibody conjugated fluorescent submicron particle suspension to the paper microfluidic chip;
(c) allowing particles and viruses spread spontaneously throughout the paper microfluidic channel via capillary action, allowing the particles to aggregate and facilitating imaging of individual particles.

8. The method of claim 7, wherein the virus is present in a concentration ranging from 10⁰ to 10⁵ virions.

9. The method of claim 7, wherein said virus is a coronavirus.

10. (canceled)

11. The method of claim 7, wherein said paper microfluidic chip comprises nitrocellulose paper, cellulose paper, or polymeric fiber filter.

12. The method of claim 7, wherein said suspension has not been pre-purified, pre-concentrated, or pre-amplified prior to testing.

13. The method of claim 7, wherein said method involves a single virus copy level detection of said virus.

14. The method of claim 9, wherein said virus is a SARS-CoV-2 virus.

15. (canceled)

16. (canceled)

17. A kit for detecting a virus comprising
a paper microfluidic chip with a pore size of about 5 μm to 15 μm depending on the type of sample suspension,
a suspension of antibody conjugated fluorescent particles (wherein the volume of the antibody conjugated fluorescent particles is from 2 uL to 6 uL and a concentration from about 0.001% to about 0.04%, depending on the type of sample suspension), and optionally a smartphone-based fluorescence microscope.

18. A kit for detecting a virus comprising
(a) a device of claim 1; and
(b) one or more reagents for carrying out detection and/or quantification of one or more coronaviruses.

19. The kit of claim 17, wherein said virus is coronavirus.

20. The method of claim 7, wherein said antibody is a polyclonal antibody or a monoclonal antibody.

21. The method of claim 7, wherein the fluorescent particle is a fluorescent polystyrene particle.

22. The method of claim 7, wherein said method further comprises:
(1) fabricating a paper microfluidic chip with multiple channels on it for simultaneously conducting multiple assays;
(2) conjugating an antibody to fluorescent particles to obtain an anti-virus antibody conjugated fluorescent submicron particle suspension;
wherein said steps are performed prior to said steps (a)-(c).

23. The method of claim 9, wherein said method further comprises:
(i) imaging the aggregation of antibody conjugated fluorescent particles;
(ii) removing background noises and autofluorescence from paper substrate using an optimized threshold intensity and isolating only the fluorescent particles;
(iii) binarizing an entire image;
(iv) removing smaller size of particles to isolate only the aggregated particles;
(v) relating sa total pixel area to the virus concentration to construct a standard curve and estimate the virus concentration from an unknown sample;
wherein said steps are performed after said steps (a)-(c).

24. (canceled)
